(19) United States
(12) Patent Application Publication
(10) Pub. No.: US 2012/0088296 A1
(43) Pub. Date: Apr. 12, 2012

(54) PHOTOBIOREACTOR SYSTEM

(76) Inventors: Jose Viriato Coelho Vargas, Curitiba (BR); Wellington Balmont, (US); Alexandre Stall, Curitiba (BR); Andre Bellin Mariano, Curitiba (BR); Juan Carlos Ordonez, Tallahassee, FL (US); Zohrob Hovsapian, Tallahassee, FL (US); Emerson Dilay, Curitiba (BR)

(21) Appl. No.: 13/271,622

(22) Filed: Oct. 12, 2011

Related U.S. Application Data
(60) Provisional application No. 61/392,053, filed on Oct. 12, 2010.

(51) Int. Cl. C12M 1/42

(52) U.S. Cl. 435/292.1

(57) ABSTRACT

A space efficient photo-bioreactor. The bioreactor grows microalgae in a tall array of transparent flooded tubes. A nutrient media is circulated through the tubes. The array is configured to maximize the amount of sunlight falling upon each tube so that growth of the microalgae is as uniform as possible. Gassing/degassing systems are attached to the array of tubes at appropriate locations. These introduce carbon dioxide and remove oxygen. Cooling systems are preferably also provided so that the circulating media can be maintained at a desired temperature. Microalgae are harvested from the photo-bioreactor. The microalgae is filtered and dried. Lipids are then extracted from the microalgae. These lipids are made into biodiesel through a trans-esterification process. The lipids may be used to make other products as well.

### CROSS-REFERENCES TO RELATED APPLICATIONS

This application is a non-provisional application claiming the benefit of an earlier-filed provisional application under 37 C.F.R.§1.53 (c). The provisional application was filed on Oct. 12, 2010. It was assigned application Ser. No. 61/392,053.

### STATEMENT REGARDING FEDERALLY SPONSORED RESEARCH OR DEVELOPMENT

None.

### MICROFICHE APPENDIX

Not Applicable

### BACKGROUND OF THE INVENTION

1. Field of the Invention

This invention relates to the field of renewable energy. More specifically, the invention comprises a space-efficient photo-bioreactor and methods for controlling the bioreactor.

2. Description of the Related Art

The continued use of petroleum-derived fuels is now widely seen as unsustainable. However, much of the existing transportation structure is dependent upon the combustion of liquid fuels. Changing to a completely different energy source—such as battery power—is at present unrealistically expensive and inefficient.

On the other hand, presently available biofuels can be substituted for petroleum-derived fuels without the need for extensively modifying existing internal combustion engines. One promising alternative fuel is biodiesel, which can be substituted for petroleum diesel in many modern engines (albeit with a slight reduction in specific energy).

Oil crops can be used to make biodiesel. These are attractive, as the total cycle of production through consumption can be made carbon-neutral. Unfortunately, though, oil crops are not very space-efficient. It is estimated that if 24% of the total cropland in the United States was devoted to a high-yielding oil crop such as palm oil, this would still only meet about half of the demand for transportation fuels.

Microalgae-based bio-fuels hold the promise of much greater space efficiency. Like plants, microalgae use sunlight to produce oils. They do it much more efficiently than crop plants, though. Microalgae-based biodiesel is still in a developmental state in terms of cost efficiency. However, it is clear that biodiesel can be made from microalgae. In order to make such a process economically efficient, it is important to use as many of the products produced as possible. The present invention proposes such a production system.

### BRIEF SUMMARY OF THE INVENTION

The present invention comprises a space efficient photo-bioreactor. The bioreactor grows microalgae in a tall array of transparent flooded tubes. A nutrient media is circulated through the tubes. The array is configured to maximize the amount of sunlight falling upon each tube so that growth of the microalgae is as uniform as possible.

Gassing/degassing systems are attached to the array of tubes at appropriate locations. These introduce carbon dioxide and remove oxygen. Cooling systems are preferably also provided so that the circulating media can be maintained at a desired temperature. The cooling system is preferably incorporated in the same units that house the gassing/degassing systems.

Microalgae are harvested from the photo-bioreactor. The microalgae is filtered and dried. Lipids are then extracted from the microalgae. These lipids are made into biodiesel through a trans-esterification process. The lipids may be used to make other products as well.

Some of the biodiesel can be used to run a diesel engine to furnish electrical and/or mechanical power to the bioreactor. Carbon dioxide emitted by the diesel engine is preferably fed back into the bioreactor. Carbon dioxide from other greenhouse gas sources is preferably also fed into the bioreactor.

### BRIEF DESCRIPTION OF THE SEVERAL VIEWS OF THE DRAWINGS

FIG. 1 is a schematic view, showing the operation of the photo-bioreactor and other related processes.

FIG. 2 is an elevation view showing the arrangement of the bioreactor tubes.

FIG. 3 is a perspective view, showing a typical circulation path for the bioreactor tubes.

FIG. 4 is an exploded perspective view, showing a typical gassing/degassing system.

### REFERENCE NUMERALS IN THE DRAWINGS

| | | | |
|---|---|---|---|
| 10 | energy harvesting system | 12 | water tank |
| 14 | nutrients | 16 | nutrient tank |
| 18 | photo-bioreactor | 20 | harvesting unit |
| 22 | filtering unit | 24 | drying unit |
| 26 | lipids extraction unit | 28 | trans-esterification unit |
| 30 | biodiesel | 32 | diesel engine |
| 34 | carbon dioxide input | 36 | inoculum input |
| 38 | support frame | 40 | rack |
| 42 | bioreactor tube | 43 | sunlight |
| 44 | elbow | 46 | gassing/degassing system |
| 48 | housing | 50 | carbon dioxide inlet |
| 52 | oxygen outlet | 54 | aluminum helix |
| 56 | coolant inlet | 58 | coolant outlet |
| 60 | inlet | 62 | outlet |

### DETAILED DESCRIPTION OF THE INVENTION

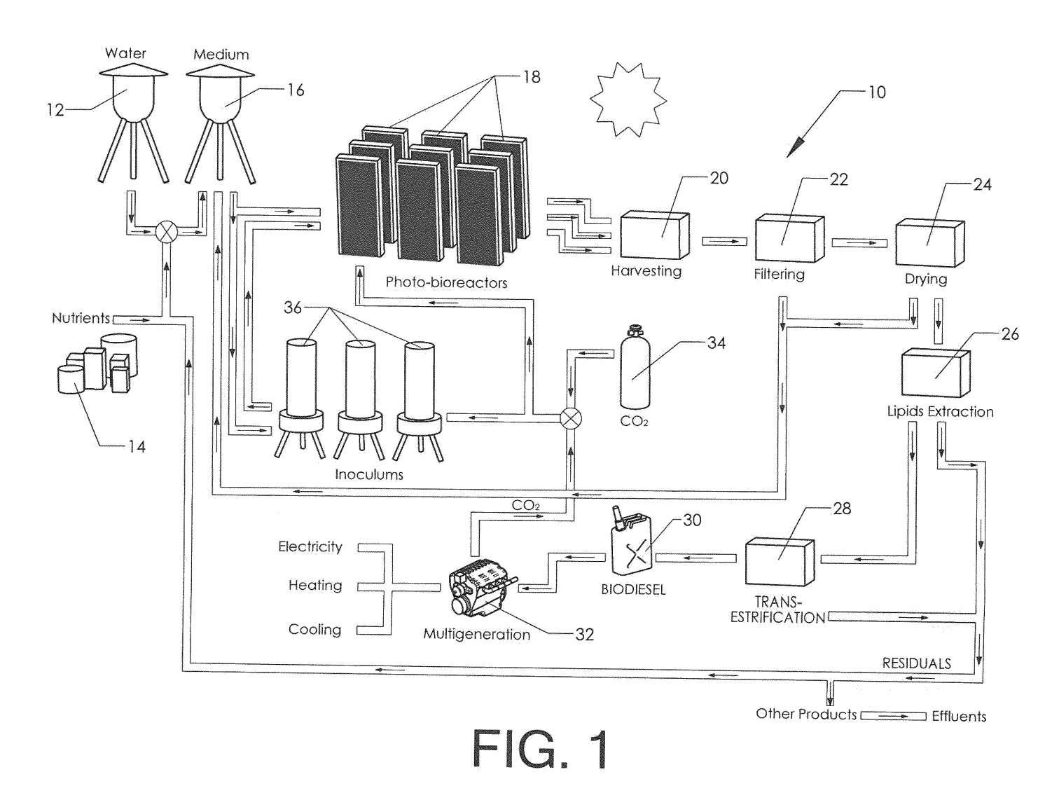

FIG. 1 shows a schematic view of a comprehensive energy harvesting system 10 based on one or more photo-bioreactors 18. The photo-bioreactors are preferably made as vertical structures having a relatively small “footprint” compared to the volume of liquid media they contain.

Nutrients 14 are mixed with water from water tank 12 (or other suitable water source) to create a nutrient medium which is preferably stored in nutrient tank 16. Inoculum input 36 is fed into a portion of the nutrient medium and this mixture is then fed into the photo-bioreactors.

Sunlight falling on the photo-bioreactors causes microalgae to grow inside. This is eventually harvested in harvesting unit 20. The product of the harvesting unit is then fed through filtering unit 22, where the microalgae is removed and residual nutrient medium is sent back to the photo-bioreactors.

The microalgae is then fed from filtering unit 22 to drying unit 24, where it is dried. The dried microalgae is then fed through lipids extraction unit 26. The extracted lipids are then sent to trans-esterification unit 28, which converts the lipids to biodiesel 30 using processes well known to those skilled in the art. The “waste” products from the lipids extraction unit are preferably fed back to the bioreactors.

The biodiesel thus produced can be transported and used as a substitute for conventional fuels. A portion of the biodiesel produced can also be used to run an on-site diesel generator. The generator can then provide power for the energy harvesting system 10.

The system preferably re-uses the products of each stage in the process. For example, the carbon dioxide produced by the on-site generator is preferably fed back into the bioreactors. More carbon dioxide will likely be needed—and this is furnished via carbon dioxide input 34.

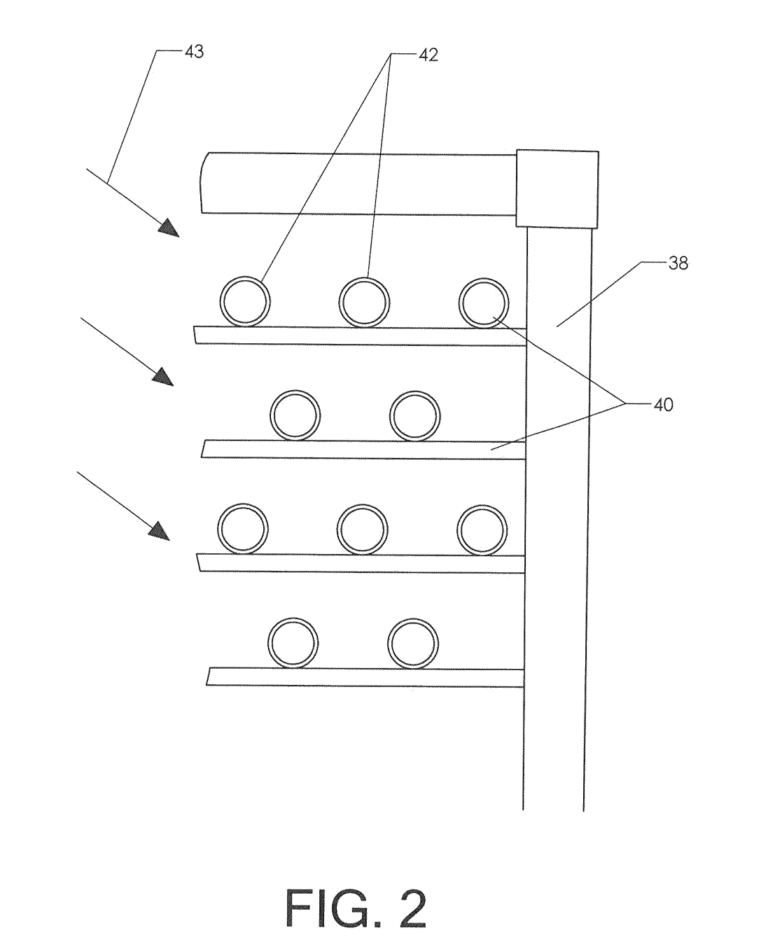

FIG. 2 shows a sectional elevation view through one of the photo-bioreactors. As mentioned previously, each photo-bioreactor preferably has a small footprint in comparison on the volume it contains. Support frame 38 supports a number of layered racks 40. Each rack 40 supports a number of bioreactor tubes 42. The tubes are relatively thin-walled transparent structures oriented perpendicularly to the view in FIG. 2. They are spaced (both horizontally and vertically) so that sunlight 43 can pass into the bioreactor and fall on each of the tubes.

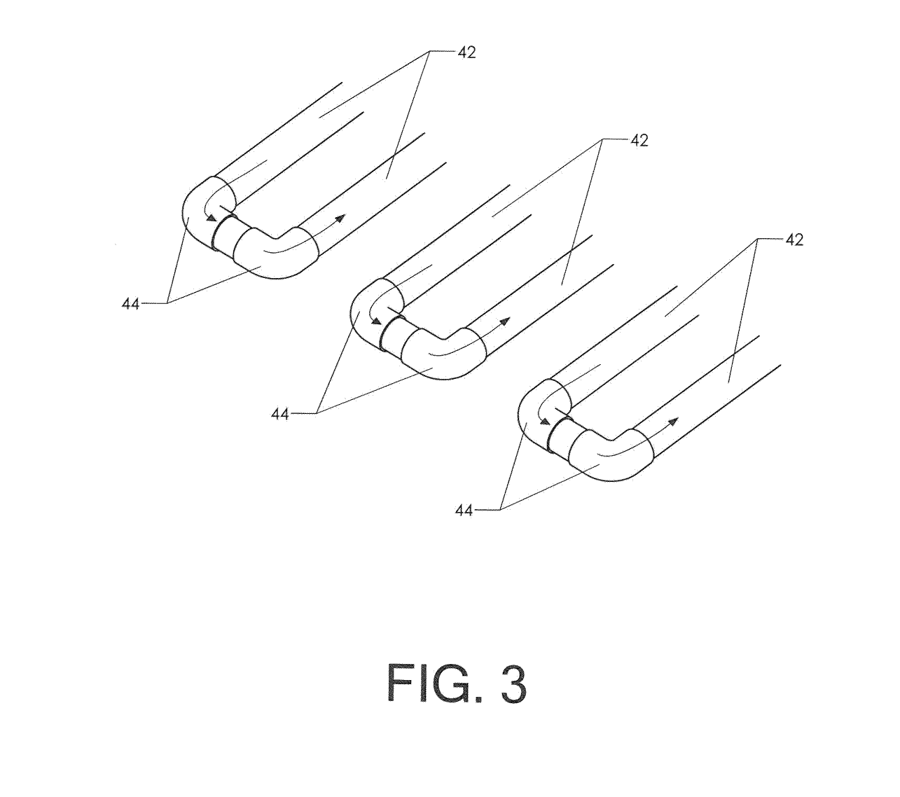

The liquid nutrient medium flows through the tubes. The tubes are joined together so that an elongated flow path is created. FIG. 3 shows one approach to joining the tubes in one rack 40. Each tube has an inlet end and an outlet end. The terms “inlet end” and “outlet end” are arbitrary terms depending on the flow direction through a particular tube. Two adjacent tubes may be joined by installing an elbow 44 between the outlet end of one tube and the inlet end of the adjacent tube. Using several such elbows a serpentine flow path can be created as in FIG. 3 (Elbows are also provided at the opposite ends of the tubes. These are not shown). Vertically oriented elbows may also be provided to join tubes on different racks 40.

It is therefore possible to create a single serpentine flow path through the entire set of tubes in a bioreactor. Of course, it may also be desirable to create two, three, or many more individual flow paths in a single bioreactor. Many different flow paths may be created, depending upon how the tubes are connected. It is also possible to use valves to create changeable flow paths. A pump is generally used to circulate the nutrient medium.

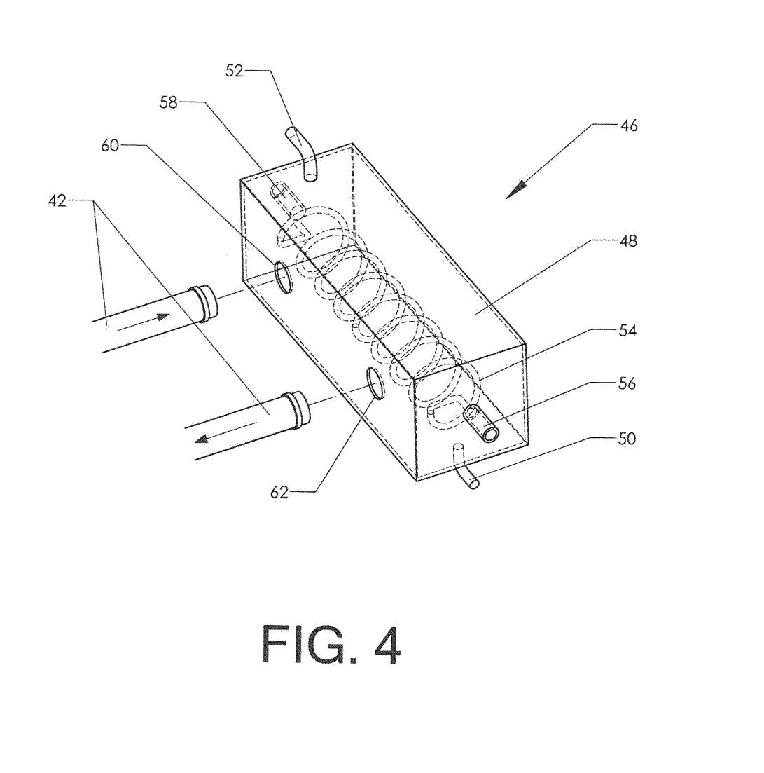

Since the microalgae growth depends on photosynthesis, carbon dioxide must be added to the circulating medium. It is also desirable to remove the oxygen produced by the photosynthesis. FIG. 4 shows a simplified depiction of a device which can provide both of these functions. Gassing/degassing system 46 has housing 48. Two bioreactor tubes 42 are connected to housing 48. Inlet flow is provided through inlet 60. Outlet flow is provided through outlet 62. Thus, the interior of housing 48 is part of a flow path within the bioreactor.

Carbon dioxide inlet 50 introduces carbon dioxide. Oxygen outlet 52 allows the escape and collection of oxygen. It is preferable to maintain the circulating medium at a desired temperature. Thus, a heat exchange device is also provided. Aluminum helix 54 is a hollow tube. Coolant inlet 56 provides inlet cooling flow through the aluminum helix. Coolant outlet carries away the coolant flow. The coolant used can be water which is cooled by a separate chiller. Other coolants may of course be used as well.

Several gassing/degassing systems 46 can be installed at suitable locations within the flow path of the bioreactor. Returning to FIG. 3, the reader will recall that simple elbows 44 may be used to direct the flow from one bioreactor tube 42 to another. Turning now to FIG. 4, those skilled in the art will realize that a gassing/degassing system 46 can be substituted for any of the elbows (with suitable adjustment being made for the distance between inlet 60 and outlet 62).

The bioreactor is largely a collection of simple components—a vertical rack with multiple horizontal tubes in an appropriately spaced location. The connections between many of the tubes will be made with elbows 44. The connection between other adjacent tubes will be made using a gassing/degassing system 46. The “control and monitoring” component is preferably part of gassing/degassing system 46. It is preferable to incorporate numerous components in housing 48. For example, the housing can contain and/or mount:
(1) carbon dioxide injecting systems;
(2) oxygen removal systems;
(3) carbon dioxide sensors;
(4) oxygen sensors;
(5) pH sensors;
(6) turbidity sensors;
(7) flow sensors; and
(8) temperature sensors.

As explained previously, the housing also preferably contains a heat exchanger capable of maintaining a desired temperature for the circulating medium. This would typically be a liquid-to-liquid heat exchanger. However—in some ambient environments—it may be possible to use a liquid-to-air exchanger. The systems for adding carbon dioxide and removing oxygen are well known in the art and will thus not be described in detail. The same may be said of the various sensors disclosed.

The reader will thus appreciate that the present invention provides a comprehensive and space-efficient system for producing biodiesel (as well as potentially other bio fuels) from microalgae. The foregoing description and drawings comprise illustrative embodiments of the present invention. Having thus described exemplary embodiments of the present invention, it should be noted by those skilled in the art that the within disclosures are exemplary only, and that various other alternatives, adaptations, and modifications may be made within the scope of the present invention. As an example, it is possible to use a gassing/degassing system 46 for every connection that is made between adjacent tubes. It is also possible to have only a single gassing/degassing system in a particular bioreactor, with all other connections being made by elbows or other suitable fittings.

Many modifications and other embodiments of the invention will come to mind to one skilled in the art to which this invention pertains having the benefit of the teachings presented in the foregoing descriptions and the associated drawings.

Having described our invention, we claim:

1. A photobioreactor for growing algae within a nutrient medium, comprising:
a. a support frame;
b. a plurality of bioreactor tubes attached to said support frame, wherein each of said bioreactor tubes has an inlet end and an outlet end;
c. a plurality of gassing/degassing housings, wherein each of said gassing/degassing housing includes,
i. an inlet connected to an outlet of a first of said bioreactor tubes,
ii. an outlet connected to an inlet of a second of said bioreactor tubes, whereby said nutrient medium flowing within said photobioreactor flows from said first bioreactor tube, through said gassing/degassing housing, and into said second bioreactor tube,
iii. a carbon dioxide inlet for injecting carbon dioxide into said nutrient medium,
iv. an oxygen outlet for removing oxygen from said nutrient medium, and
v. a heat exchanger for regulating a temperature of said nutrient medium.

2. A photobioreactor as recited in claim 1, wherein said heat exchanger is a liquid-to-liquid heat exchanger.

3. A photobioreactor as recited in claim 2, wherein said liquid-to-liquid heat exchanger is a hollow helix contained within said gassing/degassing housing, with a cooling fluid flowing through said hollow helix.

4. A photobioreactor as recited in claim 1, wherein at least some of said plurality of bioreactor tubes are joined by elbows.

5. A photobioreactor as recited in claim 1, further comprising a pH sensor in contact with said nutrient medium.

6. A photobioreactor as recited in claim 1, further comprising a temperature sensor in contact with said nutrient medium.

7. A photobioreactor as recited in claim 1, further comprising a pump for circulating said nutrient medium.

8. A photobioreactor for growing algae within a nutrient medium, comprising:
a. a support frame;
b. a plurality of horizontal bioreactor tubes attached to said support frame, wherein each of said bioreactor tubes has an inlet end and an outlet end;
c. a plurality of gassing/degassing housings, wherein each of said gassing/degassing housing includes,
i. an inlet connected to an outlet of a first of said bioreactor tubes,
ii. an outlet connected to an inlet of a second of said bioreactor tubes, whereby said nutrient medium flowing within said photobioreactor flows from said first bioreactor tube, through said gassing/degassing housing, and into said second bioreactor tube,
iii. a carbon dioxide injector,
iv. an oxygen remover, and
v. a heat exchanger.

9. A photobioreactor as recited in claim 8, wherein said heat exchanger is a liquid-to-liquid heat exchanger.

10. A photobioreactor as recited in claim 9, wherein said liquid-to-liquid heat exchanger is a hollow helix contained within said gassing/degassing housing, with a cooling fluid flowing through said hollow helix.

11. A photobioreactor as recited in claim 8, wherein at least some of said plurality of bioreactor tubes are joined by elbows.

12. A photobioreactor as recited in claim 8, further comprising a pH sensor in contact with said nutrient medium.

13. A photobioreactor as recited in claim 8, further comprising a temperature sensor in contact with said nutrient medium.

14. A photobioreactor as recited in claim 8, further comprising a pump for circulating said nutrient medium.

15. A photobioreactor for growing algae within a nutrient medium, comprising:
a. a plurality of bioreactor tubes fixedly mounted within a supporting frame, wherein each of said bioreactor tubes has an inlet end and an outlet end;
b. a plurality of gassing/degassing housings, wherein each of said gassing/degassing housing includes,
i. an inlet connected to an outlet of a first of said bioreactor tubes,
ii. an outlet connected to an inlet of a second of said bioreactor tubes, whereby said nutrient medium flowing within said photobioreactor flows from said first bioreactor tube, through said gassing/degassing housing, and into said second bioreactor tube,
iii. a carbon dioxide injector,
iv. an oxygen outlet for removing oxygen from said nutrient medium, and
v. a heat exchanger for regulating a temperature of said nutrient medium.

16. A photobioreactor as recited in claim 15, wherein said heat exchanger is a liquid-to-liquid heat exchanger.

17. A photobioreactor as recited in claim 16, wherein said liquid-to-liquid heat exchanger is a hollow helix contained within said gassing/degassing housing, with a cooling fluid flowing through said hollow helix.

18. A photobioreactor as recited in claim 15, wherein at least some of said plurality of bioreactor tubes are joined by elbows.

19. A photobioreactor as recited in claim 15, further comprising a pH sensor in contact with said nutrient medium.

20. A photobioreactor as recited in claim 15, further comprising a temperature sensor in contact with said nutrient medium.
